(19) United States
(12) Patent Application Publication
(10) Pub. No.: US 2010/0119631 A1
(43) Pub. Date: May 13, 2010

(54) Anointed body cleanser and healer

(76) Inventor: Rebecca J. Brown Johnson, Woodbridge, VA (US)

Correspondence Address: Rebecca J. Brown, 14609 Danville Rd., Woodbridge, VA, 22193, US

(21) Appl. No.: 12/588,508

(22) Filed: Oct. 17, 2009

Related U.S. Application Data
(60) Provisional application No. 61/198,703, filed on Nov. 8, 2008.

(51) Int. Cl. A61K 36/886; A61Q 19/00

(52) U.S. Cl. 424/744

(57) ABSTRACT

Anointed Body Cleanser & Healer ingredients are 100% concentrated Aloe Vera plant juice mixed with 100% distilled water.

The Aloe Vera Plants are grown fresh for the production and consumption of the Anointed Body Cleanser & Healer.

The process to produce Anointed Body Cleanser & Healer are the following ingredients: Fresh Aloe Vera Leaf & 100% Distilled Water. To produce the Anointed Body Cleanser & Healer you must grate the Aloe Vera Leaf in a blender and liquefy the leaf until it is a complete liquid that pour like water. The Aloe Vera Juice is very potent and cannot be consumed in the blended concentrated liquid form. Distilled water is needed to reduce the strength of the potency of the concentrated Aloe Vera Juice for consumption. The water is distilled by boiling for approximately 45 to 60 minutes. The distilled water must cool to room temperature.

Once the distilled water cool to room temperature the blended concentrated Aloe Vera Juice can be added to the distilled water.

The components to create the Anointed Body Cleanser & Healer is: eight (8) ounces=one (1) cup of blended concentrated Aloe Vera Juice to twenty-six (26) ounces of distilled water or three (3) cups of distilled water. This formula is the original flavor. After adding the distilled water the final product is the “Anointed Body Cleanser & Healer”.

After testing the market with the original flavor of the Anointed Body Cleanser & Healer, I will introduce to the public and society a variety of flavors such as fruit, vegetables and melons.

The Anointed Body Cleanser & Healer will be produced in glass bottle sizes ranging from two (2) ounce glass bottles to one (1) gallon jars/jugs.

Glass bottles are more appropriate and healthier than plastic bottles, jars/jugs due to the potency of the fresh concentrated Aloe Vera Juice will absorb the chemicals out of the plastic bottles, jars/jugs into the concentrated Aloe Vera Juice to be consumed by the public. The glass bottles, jars/jugs will protect and will not absorb the potency of the concentrated Aloe Vera Juice to be consumed by the public.

The purpose of Anointing Body Cleanser & Healer is to heal the body naturally without chemicals entering the body. By nature the body chemicals is suppose to be balanced at all times. Intake of chemicals through medications put the body into an position. Nature defies out-of-balance chemicals in the body and nature will make every effort to bring the body back into balance through rejecting and removing foreign chemicals from the body.

The Anointed Body Cleanser & Healer must be refrigerated after opening to prevent spoiling. The life span of the Anointed Body Cleanser & Healer is approximately one (1) year if properly refrigerated. However, if consumed properly by the recommended dosages the Anointed Body Cleanser & Healer should not be left over the life span of one (1) year.

The Anointed Body Cleanser & Healer can be purchased in concentrated form or diluted form. The concentrated form will have instructions on how to dilute the Anointed Body Cleanser & Healer for consumption.

### DESCRIPTION OF DRAWINGS

Drawings are described below as follows:

FIG. 1—2 oz. is a sample size of ABCH not for sale

FIG. 2—4 oz. is the smallest size of ABCH for sale

FIG. 3—8 oz. is the second size of ABCH for sale

FIG. 4—16 oz. is the third size of ABCH for sale

FIG. 5—24 oz. is the fourth size of ABCH for sale

FIG. 6—32 oz. is the fifth size of ABCH for sale

FIG. 7—64 oz. is the sixth size of ABCH for sale

FIG. 8—128 oz. is the seventh size of ABCH for sale

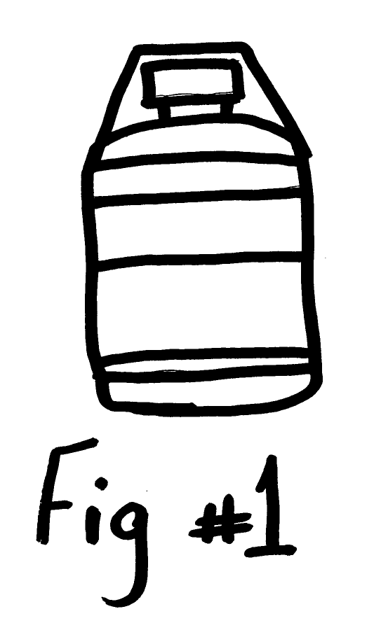

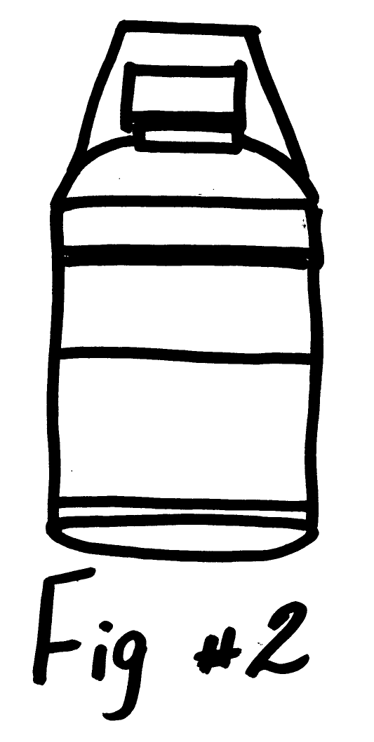

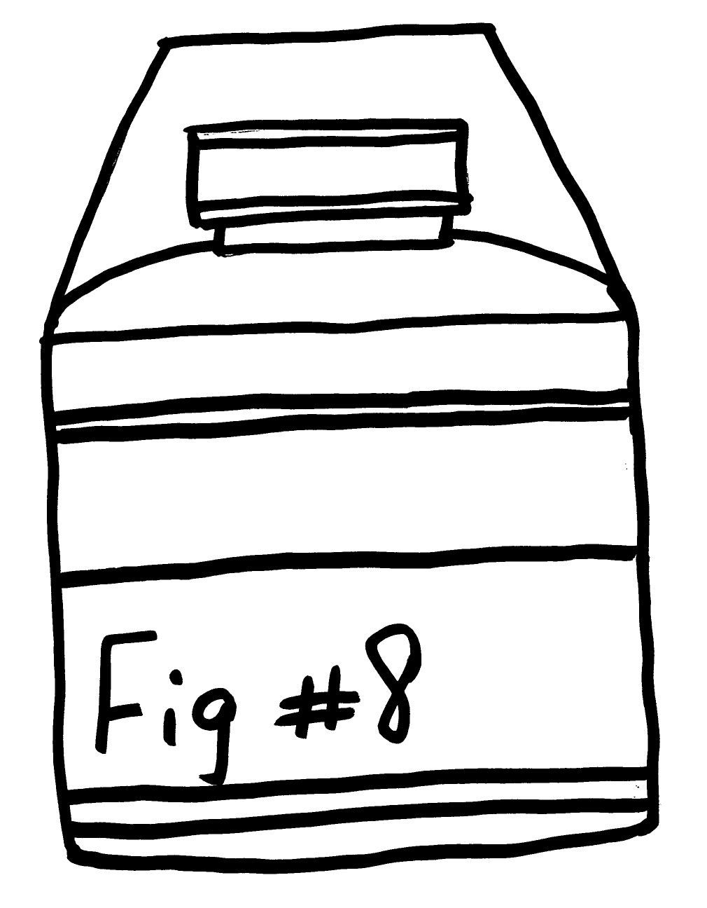

The drawings show the various sizes from the smallest to the largest sizes of bottles of ABCH will be for sale to the public and society. FIG. 1 will not be for sale it is a sample for marketing purposes only. FIG. 2 thru FIG. 8 will be for sale to the public and society based on the prices on the price list. The largest size of ABCH is FIG. 8 which is the 128 oz. or one (1) gallon jar.

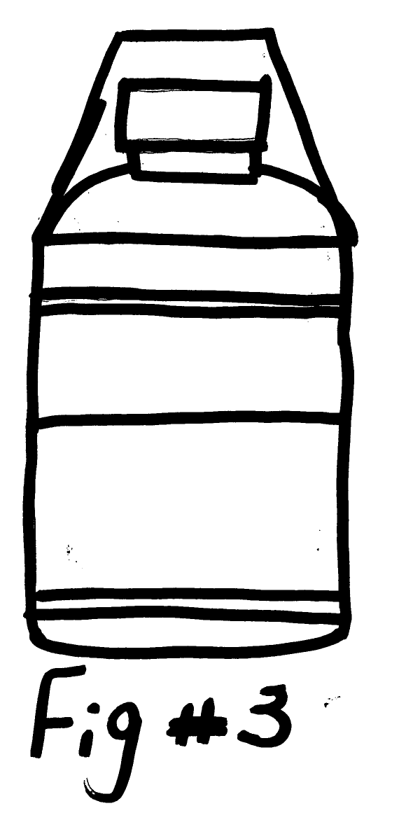

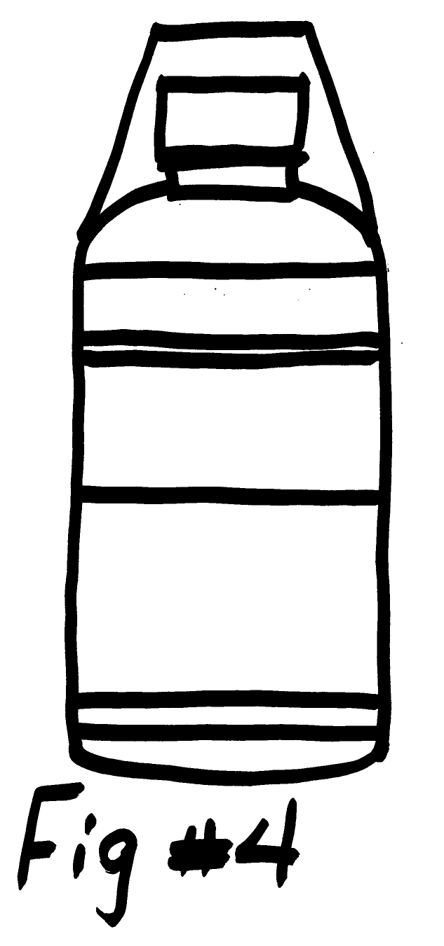

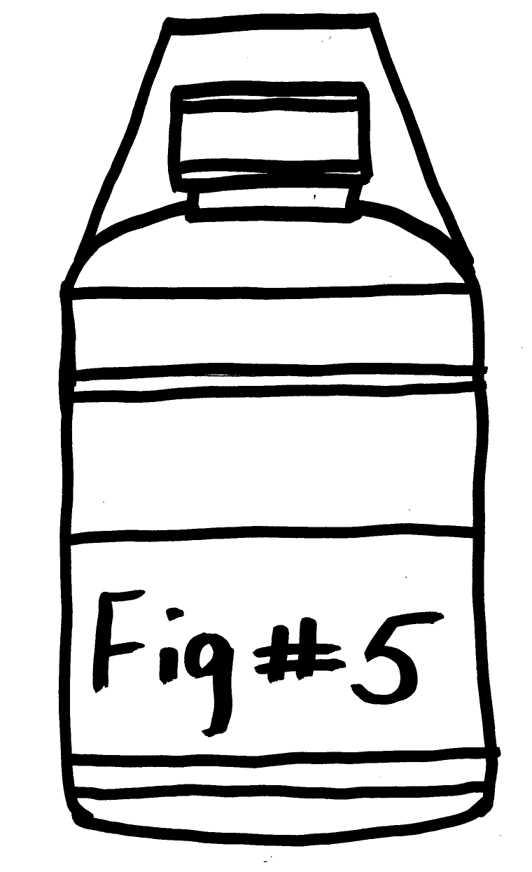

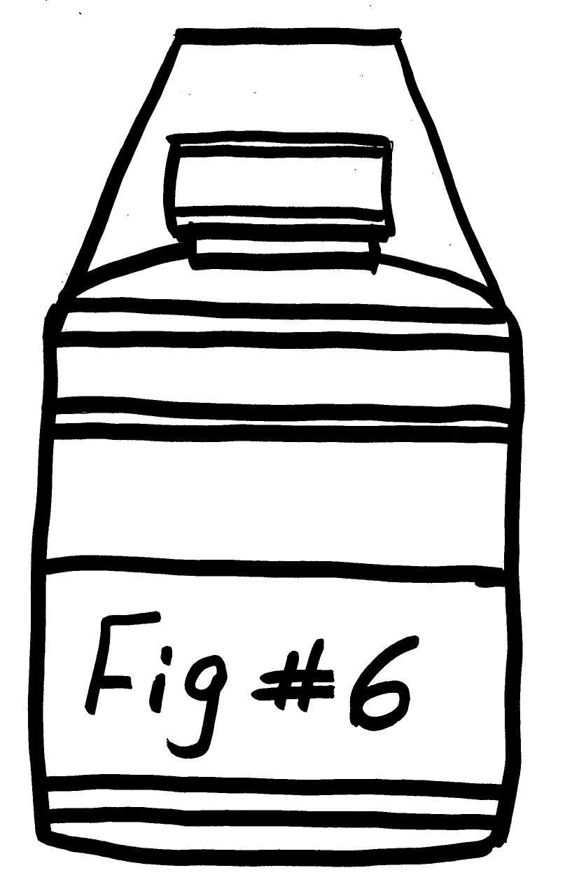

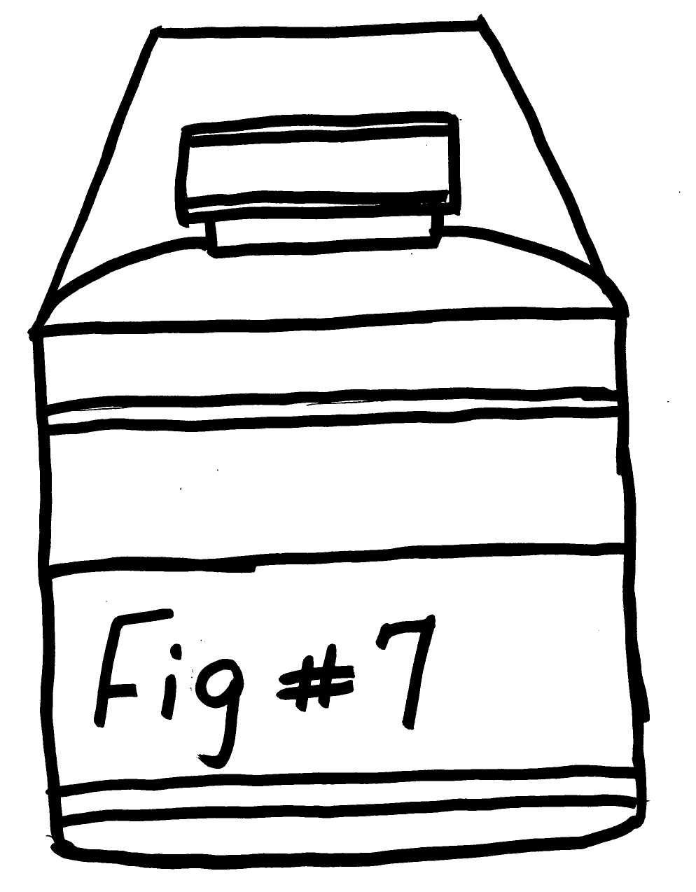

Please see the following pages of the drawings that is described in the above FIG. 1, FIG. 2, FIG. 3, FIG. 4, FIG. 5, FIG. 6, FIG. 7 and FIG. 8. Each drawing is labeled to show each size bottle separately of Anointed Body Cleanser and Healer (ABCH).

1. I claim anointed body cleanser & healer is a holistic herbal cleanser: Anointed Body Cleanser & Healer is more than a colon cleanser, it cleanse the entire body, blood and all of the body organs as well as restore needed minerals and vitamins in the body to help the body maintain it's healing abilities.
